(19) United States
(12) Patent Application Publication
(10) Pub. No.: US 2002/0072701 A1
(43) Pub. Date: Jun. 13, 2002

(54) Breast pump arrangement

(76) Inventor: Hans Ueli Nuesch, Remetschwil (CH)

Correspondence Address: Patrick C. Keane, BURNS, DOANE, SWECKER & MATHIS, L.L.P., P.O. Box 1404, Alexandria, VA, 22313-1404, US

(21) Appl. No.: 09/996,732

(22) Filed: Nov. 30, 2001

(30) Foreign Application Priority Data
Dec. 1, 2000 (DE) 100 59 710.6

(51) Int. Cl. A61M 1/06

(52) U.S. Cl. 604/74

(57) ABSTRACT

A breast pump arrangement is provided for connection to a pair of funnel-like breast receiving bodies via a corresponding pair of vacuum conduits. The vacuum conduits communicate with a vacuum source or pump. The pump unit is connected to a distribution valve to which the vacuum conduits are connected in turn. Furthermore, the distribution valve has a connection channel for the connection with the source of vacuum of the pump unit. Moreover, a regulator is provided for adjusting the suction pressure. This regulator releasably connects to the arrangement by a releasable connection. On the other hand, the regulator is suitably interposed between the source of vacuum and the connection channel.

### FIELD OF THE INVENTION

The invention relates to a breast pump arrangement for connection to a pair of funnel-like breast bodies via a corresponding pair of vacuum conduits. More particularly, it deals with a breast pump arrangement which comprises a suction pump providing a vacuum source, a distribution valve for distributing the suction to the pair of breast bodies or hoods. The distribution valve has a valve housing and connection channels for connection with the vacuum conduits. Furthermore, there is a connection piece to establish a communication between the vacuum source and the distribution valve. For adjusting the suction force, a regulator device is also provided.

### BACKGROUND OF THE INVENTION

Such a type of breast pump arrangement is on the market and corresponds approximately to an assembly of a construction according to WO 98/30257 and a regulator device according to U.S. Pat. No. 6,042,560. In this assembly, the pump, the distribution valve and the regulator device form a rigid constructive unit. The reason is, at least in part, that such a unit can easily be put as a whole onto the respective breast body and a milk collecting bottle. However, this is not so easy to be done with a design according to the above WO in comparison with that of the U.S. Pat. No. 6,042,560, because the latter was provided for one breast only and, therefore, did not need a distribution valve. In any case, the regulator device is immediately before a collection space and the milk collector bottle and was formed, in the known arrangement, by an air by-passing regulator, i.e. supplying by-passing air from ambient over a by-pass conduit. This, however, made the regulator susceptible to failure due to overflowing milk in the collecting space which made it necessary to provide relative expensive security arrangements. The collection space is normally separated from the milk collector bottle by a non-return valve.

On the other hand, manual breast pumps are also known, e.g., according to U.S. Pat. No. 4,857,051 where an electric motor pump, as in the WO document, is replaced by a manual pump and can be designed for one breast only or for both breasts.

Without a change of the respective injection molding dies and even of the whole construction, it is, however, virtually impossible to produce a similar breast pump arrangement from parts of a given arrangement to fulfill alternatively the one or the other requirement. Just regarding the different conditions and preferences of customers in different countries and for different categories of price, it would be highly desirable to produce some variants and modifications of breast pump arrangements in a cost effective manner. As mentioned above, the regulator device of the known arrangement involves a relatively expensive construction.

### SUMMARY OF THE INVENTION

Therefore, it is an object of the present invention to design a relatively cost-effective construction.

A further object of the present invention is provide a breast pump arrangement of the kind defined above which may be assembled in a flexible manner to comply with different wishes of the customer.

Another object of the present invention is to provide a breast pump arrangement of the kind described above with a regulator device which is better protected against overflowing milk without needing expensive protection arrangements.

These objects are achieved according to the invention by a breast pump arrangement in which the regulator device is releasably connected to said arrangement by removable connection means. This enables removal of the regulator device whenever adjusting the suction pressure, which acts within the breast bodies or hoods, is not desired or should be omitted for economical reasons. Moreover, it enables the regulator device to be connected selectively before or after a distribution valve. In this way, a considerable series of different models can be formed using the same components. Furthermore, replacement of individual components is enhanced.

A better protection of the regulator device, of whatever type, is provided if the regulator is interposed between the source of vacuum and the connection channel of the distributing valve. In this case, the distributing valve is nearer to the collecting space so that overflowing milk will reach it first, and it is hardly possible that it will reach the regulator. The better protection of the regulator, in turn, enables simplifying the protective measures (such as a filter, an overflow valve, labyrinth-like arrangements and so on) and, thus, leads to a more cost-effective construction. Moreover, interposing the regulator device between the distribution valve and the pump unit (in contrast to the prior art, such as U.S. Pat. Nos. 4,964,851 and 5,810,772) means separating the pump unit from the distribution valve which permits a more flexible assembly of the individual components to meet better the respective requirements of the market.

Although the regulator device may be of any type (even of that described in German Patent Application No. 197 51 018), it is preferred if it is formed as a member which influences the suction pressure (rather than suction air velocity), the suction pressure being suitably adjusted by an adjusting element accessible from outside the housing of the breast pump arrangement (although it would also be possible to have the adjusting element inside).

A suitable regulator device comprises first connection means to be releasably connected to the source of vacuum so that the regulator device could easily be removed, replaced or connected in a different way.

It is also advantageous if a distribution valve as a separate and connectable component of the breast pump arrangement is used which has a connection piece in communication with the interior of its valve housing and a releasable connection for interconnecting the suction connection channel of the breast pump unit and the connection piece. In this way, the design is more flexible, e.g., to use it selectively for a breast pump arrangement for a single breast or for both breasts.

Similarly, a pump unit according to the invention may have a suction connection channel which communicates with the vacuum source; and a releasable connection for interconnecting this suction connection channel and a connection piece of the distribution valve. This connection may be effected through a regulator device or directly according to the requirements. If the breast pump arrangement should be formed for a single breast only, no distribution valve is necessary, and the suction connection channel may be connected directly to the collecting space or to the milk collection bottle (if no collecting space is interposed).

### BRIEF DESCRIPTION OF THE DRAWING

Further details of the invention will become apparent from the following description of an especially preferred embodiment schematically illustrated in the drawings, in which:

FIG. 1 shows a perspective view of the essential parts of a breast pump arrangement according to the present invention.

FIGS. 2-4 illustrate cross-sections of the regulator device according to FIG. 1 on a larger scale, wherein FIG. 2 is a cross-section view according to the line II-II of FIG. 1, FIG. 3 is a cross-section view according to the line III-III of FIG. 1, and FIG. 4 is a cross-section view according to the line IV-IV of FIG. 2.

### DETAILED DESCRIPTION OF THE DRAWINGS

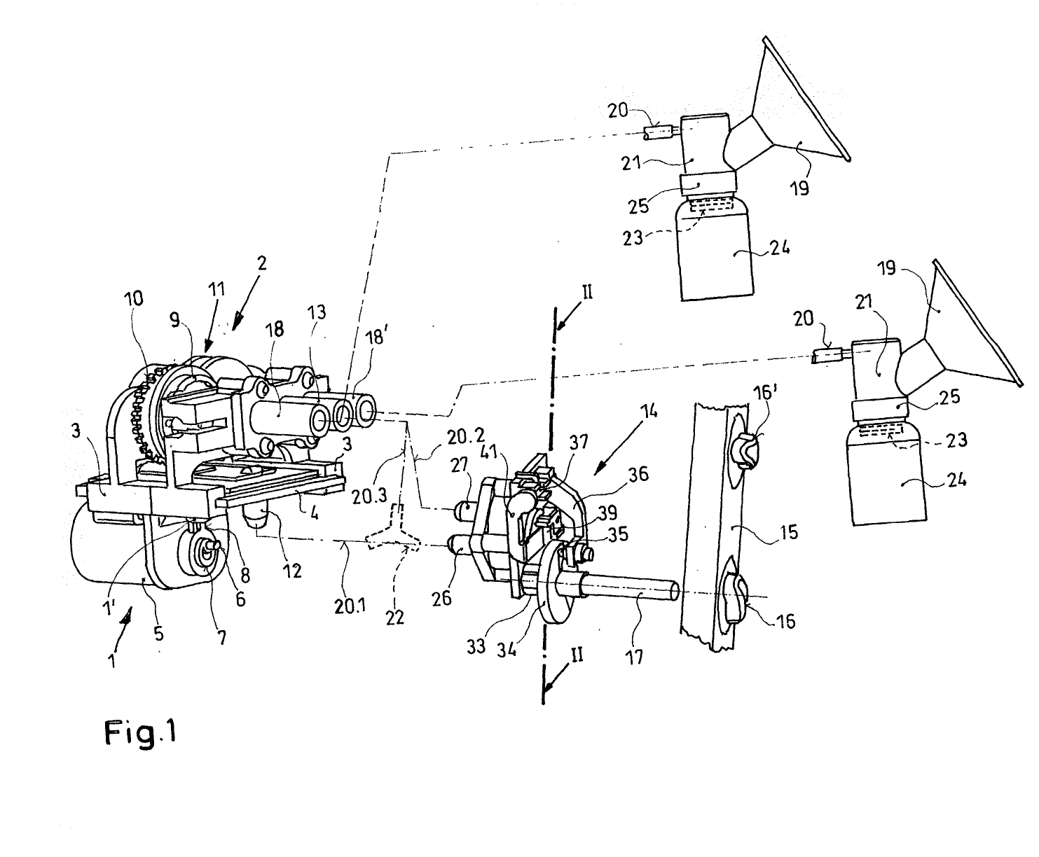

As shown in FIG. 1, a breast pump arrangement according to the invention comprises a breast pump unit 1 and a distribution valve unit 2. The valve unit 2 is secured to a frame 3 having grooves into which a hollow mounting plate 4 forming the pump cavity or (part of the) vacuum source is pushed. An electric motor 5 is fastened to this mounting plate 4 and has a motor shaft 6. On the motor shaft 6 is an eccentric 7 which drives a pump tappet 8, preferably for driving a membrane pump 1′ secured to the mounting plate 4, to perform a reciprocating pumping motion. The membrane pump 1′ together with the pump cavity in the hollow mounting plate 4 forms the vacuum source. If desired, however, any type of pump can be used, for example a plunger pump. The membrane of the pump is not visible in the view according to FIG. 1, but corresponds preferably to that of U.S. Pat. No. 5,810,772 the contents of which is hereby incorporated by reference.

In this way, the pump unit 1 may be easily removed, e.g., for repair or maintenance, by drawing it out of the frame 3. Thus, it is possible, if desired, to combine a manual pump, a plunger pump or any other pump with the remaining components of the breast pump arrangement according to the invention, such as with the distribution valve unit 2. However, it is to be understood that the frame 3 may be part of a housing and may comprise a further groove for inserting another mounting plate which carries the valve unit (or any other part).

The valve unit 2 may be of any kind, for example of that one which is disclosed in U.S. Pat. No. 5,810,772 mentioned above or of that one described in WO 98/30257. In any case, a design is preferred which includes reciprocating valve members rather than rotating ones, as disclosed in U.S. Pat. No. 4,964,851. For actuating at least one valve member, a cam disk 9 is provided, as is known per se, which is driven either by a motor for its own, as suggested in U.S. Pat. No. 6,045,529, or by engaging a motor pinion of the pumping unit 1 with a gear wheel 10 of a reduction gear mechanism 11 for driving the cam disk 9. International PCT Publication WO 98/30257, U.S. Pat. No. 4,964,851 and U.S. Pat. No. 6,045,529 are hereby incorporated by reference.

The pump unit 1 has a suction connection channel 12 leading out of the interior of the hollow mounting plate 4, i.e., out of the pumping space. A connection hose 20.1 may be put up onto this suction connection channel 12. This connection hose 20.1 communicates with another connection hose 20.2 which leads to the center between two valve chambers (not shown) of the valve unit 2, i.e., through a center connection channel 13. Normally, i.e., according to the prior art, the parts 12 and 13 would communicate directly. However, by using a releasable connection, in this embodiment formed by the connection hoses 20.1 and 20.2, the units 1 and 2 may be separated from one another in order to enable a modular construction.

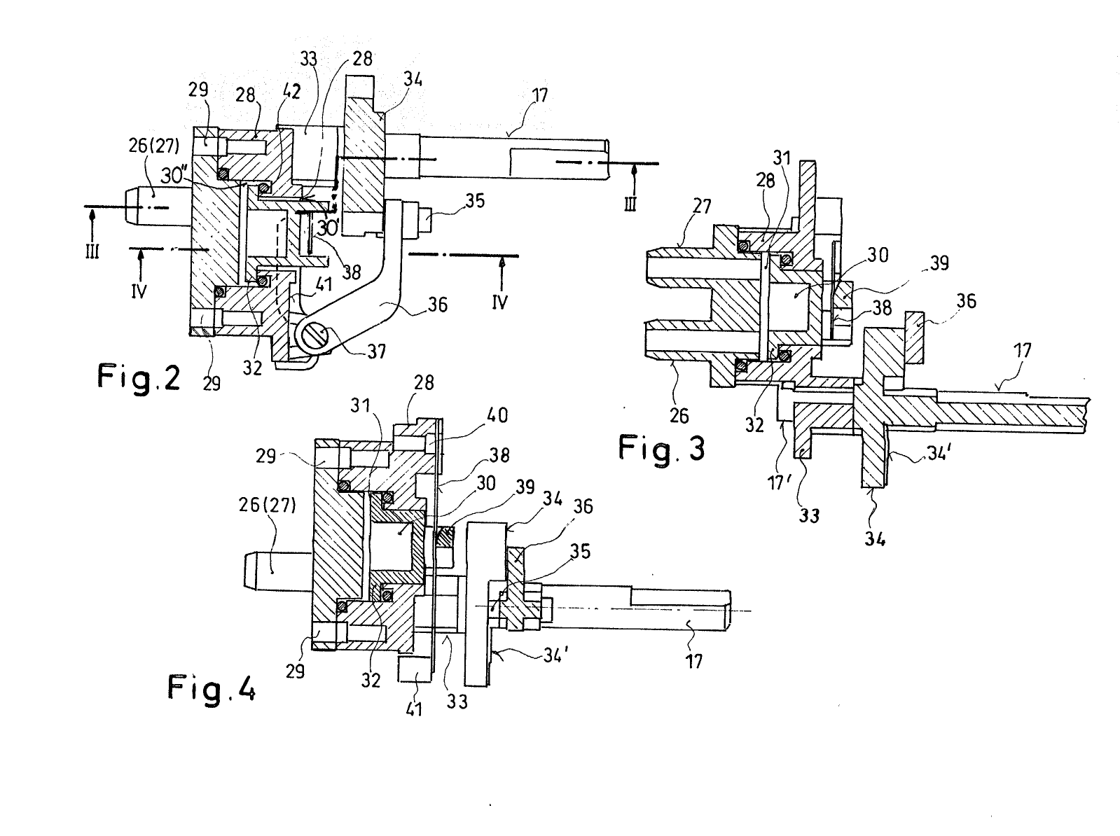

Moreover, it may be seen that it would also be possible to establish the normal function using a single connection hose 20.1 or 20.2 directly between the parts 12 and 13 for transferring the suction pressure prevailing at the suction connection channel 12 to the connection channel 13. In the present case, however, a regulating device 14 is interposed which will be described in detail with reference to FIGS. 2 to 4. This regulating device 14 is preferably situated in proximity of a housing's wall 15 in order to enable adjusting from outside by means of an adjusting knob 16 fastened to an adjusting shaft 17 of the regulating device 14. Although this arrangement is preferred, it should be noted that the regulating device may, alternatively, be formed as a mere pre-regulating device for pre-setting a desirable suction pressure before delivering the breast pump arrangement according to the invention to a shop or to a customer. In this case, the adjusting knob 16 might be omitted, while the adjusting shaft 17 may not extend through the housing's wall 15, may be shorter than illustrated and may be set only by the personnel of a shop after opening the housing. Of course, the adjusting knob can also be omitted, if a regulator device 14, in a simpler embodiment of the breast pump arrangement according to the invention, should be omitted as a whole, and instead of the hoses 20.1, 20.2 there is a single short circuiting hose between the channels 12 and 13, as mentioned above. Another knob 16′ may serve to switch the motor 5 on. It will be understood by those skilled in the art that the invention is not restricted to using a turning knob, but sliders, keys or any other adjusting or setting element could be used.

The regulating device 14 has an entrance connection 26 which communicates with the suction connection channel 12 of the pump unit 1 through the connection hose 20.1, and a further connection piece 27 connected to the connection channel 13 of the valve unit 2. Moreover, the valve unit 2 has a connection or outlet bore 18, 18′, as known per se, each leading through a corresponding hose 20 to a suction chamber 21 and to the respective breast receiving body 19. A milk collecting bottle 24 communicates with the suction chamber 21 via a non-return valve 23 for closing a suction space 25 in one direction.

Although the regulator device 14 may be formed in any way desired, e.g., as a throttle valve as disclosed in German Patent Application 197 51 018, the embodiment shown in FIGS. 2 to 4 is preferred, which is, still more preferred, formed as an air by-passing regulator. Particularly in FIG. 3 may be seen how the two connection pieces 26, 27 communicate with each other. Both connections orifice into a regulator housing

is opposite a pot-like adjusting member 30 and is connected to them via screws, pegs or pins 29 (FIG. 2). This adjusting member 30 is movable within an at least approximately cylindrical space 31 (although, in principle, any cross-section could be used), i.e., between a lefthand end position and a right-hand end position (with reference to FIGS. 2 to 4). The setting member has a rim or flange 32 which, according to FIG. 3 is so dimensioned that it is able to cover the orifices of the connections 26 and 27 at least in part. It should be noted that, for reasons of symmetry, it is preferred, if the rim 32 can cover both orifices, but it would be sufficient to fulfill the function as an adjustable flow resistance (which the setting member is in this embodiment), if the rim or flange 32 covered only one of the orifices of the connections 26 and 27. If the rim 32 covers both orifices in the lefthand end position shown of the adjusting member 30, any suction may be prevented (in which case the regulating device 14 operates as a throttle valve, which is not preferred). In the right-hand end position, in contrast, both orifices communicate with each other. Any intermediate position reduces the flow cross-section.

If, as mentioned above, it is desired to set the regulator device by a man skilled in the art only before delivering the pump arrangement to a customer or to perform after-adjustments only by such a man, the right-hand end position of the adjusting member 30 could, for example, be defined by an adjustable stop, such as an adjusting screw engaging the right-hand outer surface of the pot-like adjusting member 30. However, in the preferred embodiment (as shown), the housing 28 has a bearing projection 33 (see FIG. 3), in which one end 17′ of the adjusting shaft 17 is rotatably supported. The adjusting shaft 17 has an adjusting cam 34 secured to it, as best seen in FIGS. 1, 3 and 4.

The cam surface 34′ of the adjusting cam 34 is engaged by a cam follower pin 35, as seen particularly in FIG. 1. This cam follower 35, according to FIGS. 1 and 2, is mounted to the free end of an adjusting lever 36, the other end of which is secured to a shaft 37. In principle, one could provide the shaft 37 with an end stop for a desired end position of the adjusting member 30. In the present preferred embodiment, however, the position of the adjusting member 30 is determined, by the force of a spring opposing the force of the suction pressure (negative pressure) between the orifices of the connections 26 and 27. This spring could be formed in any way desired, be it as a pressure spring or as a tension spring, but the problem is that the available space in appliances, such as the present breast pump arrangement, is very small. Therefore, it has been found advantageous to form the spring as a leaf spring 38.

According to FIGS. 2 to 4, suction in the cylindrical space 31 acts in the sense of a movement of the adjusting member 30 to the left. The adjusting member 30 has, as is clearly seen particularly in FIG. 3, a strap 39 which overlaps the leaf spring at the adjusting member 30 is practically elastically suspended by the leaf spring 38. One end of the leaf spring 38 firmly secured to the housing 28, e.g., by a screw 40 (FIG. 4) or in any way desired. The other, free end of the leaf spring

a lever 41 (FIG. 1, 2) in the manner shown in FIGS. 1 and 4. Thus, the rotational position of the lever 41 determines the force of the spring

acts onto the strap 39 and, thus onto the adjusting member 30. The rotational position of the lever 41 is also determined, as illustrated in FIGS. 1 and 2, by the fact that it is secured to the shaft 37 to which the adjusting lever 36 is firmly secured.

Therefore, when turning the adjusting cam 34 by means of the shaft 17, the cam follower 35, which engages the cam surface, is shifted in accordance with the axial thickness of the cam. The cam follower 35, however, engages the axial curve of the cam surface 34′ under the force of the leaf spring 38. Thus, the leaf spring 38 double function, i.e., biasing the adjusting member 30 to the right, and pressing the cam follower 35 to the cam surface 34′. This double function is fulfilled in that the leaf spring

a rotational moment onto the lever 41 secured to the shaft 37 and, thus, also to the adjusting lever 36 which supports the cam follower 35.

According to the above description with reference to FIGS. 2 and 4, an adjusting member was supposed whose rim 32 is cylindrical and fills sealingly the whole diameter of the cylindrical space 31. However, the regulating device can easily operate as an air by-passing regulator, as is preferred, if the adjusting member 30 is shaped in such a way that defines a gap together with the bore wall 28′ of the housing 28(FIG. 2), the gap extending at least (and preferably only) over part of the outer circumference of the pot-like adjusting member 30, thus forming an air by-pass conduit.

Similarly, the rim 32 does not sealingly engage the cylindrical wall of the space 31, but leaves a further free gap 30″ which may communicate with the gap 30′. In the right-hand end position shown in FIG. 2, the rim 32 engages a sealing ring 42 SO that the air by-pass conduit through the bore 30′ is shut. Only when the adjusting member 30 moves from the position shown to the left, this air by-pass conduit is opened more or less. When using such an air by-passing construction, it would also be conceivable to use a space 31 which enlarges slightly to the left, e.g., being conical rather than cylindrical. This arrangement and its modifications, however, as have just been described with reference to FIG. 2, facilitate a flexible adaptation of the breast pump arrangement according to the invention to different requirements. When used as an air by-pass regulator, it will be understood that the negative pressure of the suction on the left side of the adjusting member 30 is balanced by the force of the leaf spring 38 which is adjustable in the manner described.

For example, an air by-pass regulating device 14 of the type described above could be used in which, however, the connection 27 (FIG. 1) is either omitted or stopped up. The influence of the suction pressure by the flow of by-passed air acts immediately onto a Y-piece 22, merely represented in dotted lines, which effects a connection in parallel of the regulator device 14 and the valve unit 2, i.e., the by-passed air flows to a suction path formed by the conduit 20.1, on the one hand, and another conduit 20.3, on the other hand, which leads directly to the connection channel 13. Such an embodiment would protect the regulating device 14 against entering of milk, even if milk passed the valve unit 2 in the direction towards the pump unit 1, because the flow of by-passed air would be opposite to such flow of milk. This may be of advantage, because regulator units are normally susceptible to contamination by milk so that such an arrangement would provide additional protection. On the other hand, it is clear that such additional protection is paid by the additional price of the Y-piece 22 and the additional hose connection to the connection tube 26. For this reason, the first mentioned variant of an air bypassing regulator comprising the hose 20.2 will be preferred in all cases where the risk of contamination can be banned by other constructive measures. Moreover, this risk is relative small due to the fact that milk cannot reach the regulator 14 directly, but only via the valve unit 2.

Numerous modifications are possible within the scope of the invention; for example, the cylindrical space 31 could be enlarged to the right so that suction, which prevails in the hoses 20.1 and 20.2, is directed to the right side of the adjusting member 30. In this case, the spring

be arranged in such a way that it biases the adjusting member 30 towards the lefthand end position in which it restricts the flow between the hoses 20.1 and 20.2 and increases the flow resistance. However, it will be understandable to those skilled in the art that the construction shown is simpler.

Instead of a single leaf spring 38, a plurality of such leaf springs, for example as a spring assembly, could be used, e.g., for providing the spring with a desired characteristic.

Furthermore, instead of an axial cam, as shown, a radial cam could be used either for adjusting the end position of the adjusting member 30 or the force of the spring 38. Nevertheless, an axial cam 34, 34′ is more favorable, because it needs less space.

It has been mentioned, how a releasable connection between the individual components 1, 2 or 14 can be accomplished by means of hoses 20.1, 20.2, 20.3. Of course, a releasable connection could also be achieved by solid conduit elements interconnected via couplings or flange connections.

It will also be understood that the adjusting member 30, in the case of its function as an adjustable flow resistance, could be formed in various ways. For example, a slider could be used which has openings or nozzles of different cross-sectional area, one of them being inserted into the path of flow. Another alternative could be formed by a valve needle shiftable within a nozzle opening to vary its effective cross-section. Other alternatives are known to those skilled in the art of valve construction.

What is claimed is:

1. A breast pump arrangement for connection to a pair of funnel-like breast receiving bodies via a corresponding pair of vacuum conduits, the arrangement comprising:
a source of vacuum including pumping means;
distribution valve means including:
connection means for connection with said pair of funnel-like breast receiving bodies, and
connection channel means for connection with said source of vacuum, so that vacuum produced by said source of vacuum is distributed to said connection means to exert a suction pressure; and
regulator means for adjusting said suction pressure, said regulator means being releasably connected to said arrangement by releasable connection means.

2. An arrangement as claimed in claim 1, wherein said removable connection means comprise at least one hose.

3. An arrangement as claimed in claim 1, further comprising housing means and manual setting means connected to said regulator means, said manual setting means being accessible from outside said housing means.

4. A breast pump arrangement for connection to a pair of funnel-like breast receiving bodies via a corresponding pair of vacuum conduits, the arrangement comprising:
a source of vacuum including pumping means;
distribution valve means including:
connection means for connection with said pair of funnel-like breast receiving bodies, and
connection channel means for connection with said source of vacuum, so that the vacuum produced by said source of vacuum is distributed to said connection means to exert a suction pressure in said breast receiving bodies;
regulator means for adjusting said suction pressure, said regulator means being interposed between said source of vacuum and said connection channel means.

5. Regulator device as claimed in claim 4, wherein said first connection means form the only connection to be connected both with said source of vacuum and said distribution valve means in parallel.

6. Regulator device as claimed in claim 4, further comprising an Y-shaped tube having three branches, one being connected to said first connection means, a second one being connected to said source of vacuum, and a third one being connected to said distribution valve.

7. A regulator device for a breast pump arrangement having a source of vacuum for providing suction of a predetermined suction pressure in a connection channel of distribution valve means for suctioning milk from two breasts, the device comprising:
an air conduit system leading to a connection channel, said air conduit system having a predetermined cross-section;
adjusting means for adjusting suction pressure acting on the breasts, said adjusting means being movable between two opposite end positions, and
first connection means for releasable connection to said source of vacuum.

8. Regulator device as claimed in claim 7, further comprising second connection means for connection to said connection channel of said distribution valve means.

9. Regulator device as claimed in claim 8, wherein said adjusting means comprise an adjusting element interposed between said first and second connection means and being movable between said end positions for varying the transfer of a vacuum from said first connection means to said second connection means.

10. Regulator device as claimed in claim 7, further comprising an air by-pass conduit of predetermined cross-section for by-passing air to said air conduit system and, thus, influencing said suction pressure, said adjusting means comprising an adjusting element in said by-pass conduit for varying an effective cross-section of said by-pass conduit.

11. Regulator device as claimed in claim 10, further comprising second connection means for connection to said connection channel of said distribution valve means, said adjusting element being interposed between said first and second connection means.

12. Regulator device as claimed in claim 7, wherein said adjusting means comprise an adjusting element in said air conduit system for varying an effective cross-section of said air conduit system, one of said end positions corresponding to a maximum effective cross-section.

13. Regulator device as claimed in claim 12, wherein said adjusting means comprise an adjusting element in said air conduit system for varying an effective cross-section of said air conduit system, another of said end positions corresponding to minimum effective cross-section.

14. Regulator device as claimed in claim 7, further comprising passage means for communicating said first connection means with the atmosphere for leaking atmospheric air into said first connection means through an effective leaking cross-section, said adjusting means being adapted to vary said leaking cross-section.

15. A regulator device for a breast pump arrangement having a source of vacuum for providing suction of a certain suction pressure in a connection channel of distribution valve means for suctioning milk from two breasts, the device comprising:
an air conduit system leading to a connection channel, said air conduit system having a predetermined cross-section;
adjusting means for adjusting suction pressure acting on the breasts, said adjusting means being movable between two opposite end positions and comprising spring means exerting a spring force for biasing said adjusting means into one of said two end positions; and
first connection means for connection to said source of vacuum.

16. Regulator device as claimed in claim 15, wherein said spring means comprise at least one elongated leaf spring having two ends.

17. Regulator device as claimed in claim 15, wherein said adjusting means comprise a setting means for varying said spring force.

18. Regulator device as claimed in claim 17, wherein said spring means comprise at least one elongated leaf spring having two ends, said adjusting means engaging said leaf spring between its two ends, while said setting means engage one end of said leaf spring.

19. Regulator device as claimed in claim 17, wherein said adjusting means comprise an adjusting element, said setting means comprise cam means of a predetermined cam shape and being movable into different positions, cam follower means for following the shape of said cam means, and transfer means connected to said cam follower means for transferring said shape to said adjusting element in accordance with the position of said setting means.

20. Regulator device as claimed in claim 19, wherein said transfer means comprise first lever means having a free end connected to said cam follower means, and second lever means connected to said first lever means engaging said adjusting element.

21. Regulator device as claimed in claim 19, wherein said cam means comprise an axial cam.

22. A distribution valve breast pump arrangement for connection to a pair of funnel-like breast receiving bodies via a corresponding pair of vacuum conduits, said breast pump arrangement including a breast pump unit which forms a source of vacuum for providing suction of a given suction pressure in a suction connection channel for suctioning milk from two breasts, said distribution valve forming a unit separate from said breast pump unit, the distribution valve comprising:
valve housing means forming a hollow interior;
a connection piece in communication with said hollow interior; and
releasable connection means for interconnecting a suction connection channel of a breast pump unit and said connection piece.

23. A breast pump unit forming a vacuum source for a breast pump arrangement for connection to a pair of funnel-like breast receiving bodies via a corresponding pair of vacuum conduits via a distribution valve having a connection piece for connection with the breast pump unit, the breast pump unit being separate from and releasably connected to said distribution valve, said breast pump unit comprising:
a valve housing accommodating a vacuum source;
a suction connection channel communicating with said vacuum source; and
releasable connection means for interconnecting said suction connection channel and a connection piece of a distribution valve.

24. Breast pump unit as claimed in claim 23, wherein said releasable connection means comprise hose means.
